(19) United States
(12) Patent Application Publication
(10) Pub. No.: US 2024/0377056 A1
(43) Pub. Date: Nov. 14, 2024

(54) DISINFECTING LIGHTING DEVICE WITH IMPROVED SAFETY AND LIGHTING PERCEPTION

(71) Applicant: SIGNIFY HOLDING B.V., EINDHOVEN (NL)

(72) Inventors: TIES VAN BOMMEL, HORST (NL); ERIK PETRUS JOHANNES MALLENS, 'S-HERTOGENBOSCH (NL)

(21) Appl. No.: 18/559,091

(22) PCT Filed: Apr. 25, 2022
(86) PCT No.: PCT/EP2022/060804

§ 371 Date: Nov. 6, 2023

(30) Foreign Application Priority Data
May 7, 2021 (EP) 21172754.0

(51) Int. Cl. F21V 33/00; A61L 9/20; F21Y 113/00; F21Y 113/10; F21Y 115/10

(52) U.S. Cl. CPC F21V 33/0064; A61L 9/20; A61L 2209/12; A61L 2209/212; F21V 2200/20; F21Y 2113/10; F21Y 2113/30; F21Y 2115/10

(57) ABSTRACT

The present invention relates to a disinfecting lighting device (1) arranged to emit device light. The disinfecting lighting device comprises at least one disinfecting light module (2, 2′) comprising at least one first light source (3, 3′) providing a first light in the UV wavelength range and a second light in the visible wavelength range, the second light having a first color point x₁,y₁. The disinfecting lighting device further comprises at least one second light source (4) configured to provide a third light in the visible wavelength range, the third light having a second color point x₂,y₂. An optical element (5) is arranged in a light-receiving relationship with the at least one disinfecting light module (2, 2′) and the second light source (4). The first color point xi,yi and the second color point x₂,y₂ are substantially equivalent.

### TECHNICAL FIELD

The present invention relates to a disinfecting lighting device having improved performance in term of lighting perception and safety.

### BACKGROUND

In view of the recent development in the world concerning the global pandemic, disinfection has become a topic of renewed interest as the demand for sterilization increases. One way of disinfecting involves the use of UV light. As a response to pathogenic outbreaks involving airborne microorganisms it would be beneficial to employ UV light for disinfecting air and objects at locations where the transmission of such microorganisms is believed to occur.

Disinfecting luminaires are used to flood spaces such as hospital rooms with UV-B (ultra-violet light of 280-315 nanometer (nm)) and UV-C (ultra-violet light of 200-280 nm) radiation for disinfection purposes. Such disinfecting luminaires require a relatively brief time, e.g. several minutes, to achieve adequate disinfection but require the room to be evacuated of people since the irradiation is harmful to human skin and eyes.

Normally, disinfection by UV light sources is used under controlled conditions in areas where humans or animals are not present during ongoing disinfection, such as at surgery theaters or the like. However, the increased demand for germicidal activities may involve operating UV light sources in environments with human presence, thus introducing a risk for unintentional irradiation by UV light. Therefore, disinfecting light sources, in particular those involving UV light, should possess reliable safety features in order to avoid potential exposure of humans or animals to the harmful irradiation.

It has been shown that far-UVC light (190-230 nm) efficiently kills pathogens most likely without harm to exposed human tissues. Continuous far-UVC exposure in occupied public locations at the current regulatory exposure limit (˜3 mJ/cm²/hour) would result in ˜90% viral inactivation in ˜8 minutes, 95% in ˜11 minutes, 99% in ˜16 minutes and 99.9% inactivation in ˜25 minutes. Thus, while staying within current regulatory dose limits, low-dose-rate far-UVC exposure can potentially safely provide a major reduction in the ambient level of airborne microorganisms in occupied public locations (Buonanno et al., Sci Rep 10, 10285, 2020).

However, availability of disinfecting lighting devices utilizing far-UVC is still rather limited. Further, far-UVC lighting devices suffer from the disadvantage of poor lighting perception, wherein the far-UVC light sources appear as dots or bright areas, while other portions of the light-emitting surface are not illuminated at all.

In view of the above, it is desired to obtain a disinfecting lighting device having a high level of safety and improved optical performance compared to the solutions suggested by the prior art.

### SUMMARY

The present invention thus provides such a disinfecting lighting device. The disinfecting lighting device of the present invention may be particularly suitable for disinfecting spaces with high level of activity, such as a waiting room in a hospital or a veterinary clinic, a public space such as a library, an office, a department store or the like, an office space, as well as public transportation means, such as busses or trains.

The disinfecting lighting device according to the present invention is arranged to emit device light and comprises at least one disinfecting light module comprising at least one first light source providing a first light in the UV wavelength range and a second light in the visible wavelength range. The second light has a first color point x₁,y₁. The UV wavelength range may be from 100 nm to 380 nm (i.e. UVC, UVB, UVA), and preferably from 100 nm to 285 nm (i.e. UV-C). In particular, the UV wavelength range may be from 190 nm to 230 nm (i.e. far-UVC). Preferably, the wavelength of the first light is 207 nm, 222 nm, or combination thereof. By the term “visible wavelength range” in the context of the present invention is understood a wavelength range from 380 nm to 750 nm.

The ultraviolet wavelength range is defined as light in a wavelength range from 100 to 380 nm and can be divided into different types of UV light/UV wavelength ranges. With reference to Table 1, different UV wavelengths of radiation may have different properties and thus may have different compatibility with human presence and may have different effects when used for disinfection. In Table 1, a “+” sign indicates that light in the specific range has the indicated effect, while a “−” sign indicates that the light in the specific range does not have the indicated effect. A “+/−” sign indicates a moderate effect. As mentioned below, the effect may in itself be desired or undesired (e.g. ozone generation).

TABLE 1
Properties of different types of UV wavelength light

| | | | (Relative) | | | | |
|---|---|---|---|---|---|---|---|
| | | | sterilization | | | | |
| | Short | Wavelength | effectiveness | | Safe | Vitamin D | Ozone |
| Name | name | (nm) | Bacteria | Viruses | Radiation | generation | generation |
| Violet | V | 380-420 | +/− | − | + | | |
| Ultra-violet A | UV-A | 315-380 | + | − | + | | |
| Ultra-violet B | UV-B | 280-315 | + | +/− | +/− | + | |
| Near ultra- | Near | 230-280 | + | + | − | | |
| violet C | UV-C | | | | | | |
| Far ultra-violet | Far UV | 190-230 | + | + | + | | +/− |
| Extreme ultra- | Extreme | 100-190 | + | + | − | | + |
| violet C | UV-C | | | | | | |

Each UV type/wavelength range may have different benefits and/or drawbacks. Relevant aspects may be (relative) sterilization effectiveness, safety (regarding radiation), and ozone production (as result of its radiation). Depending on an application, a specific type of UV light or a specific combination of UV light types may be selected and provides a superior performance over other types of UV light. UV-A may be (relatively) safe and may kill bacteria but may be less effective in killing viruses. UV-B may be (relatively) safe when a low dose (i.e. low exposure time and/or low intensity) is used, may kill bacteria, and may be moderately effective in killing viruses. UV-B may also have the additional benefit of production of vitamin D in a skin of a person or animal. Near UV-C may be relatively unsafe but may effectively kill bacteria and viruses. Far UV may also be effective in killing bacteria and viruses but may be (relatively to other UV-C wavelength ranges) (rather) safe. Far-UV light may generate some ozone which may be harmful for human beings and animals. Extreme UV-C may also be effective in killing bacteria and viruses but may be relatively unsafe. Extreme UV-C may generate ozone which may be undesired when coming in contact with human beings or animals. In some application, ozone may be desired and may contribute to disinfection, but then shielding of humans and animals may be desired. Hence, in the table “+” for ozone production especially implies that ozone is produced which may be useful for disinfection applications but may be harmful for humans/animals when they are exposed to it. Hence, in many applications this “+” may actually be undesired while in others, it may be desired.

The first light source may be a solid-state light source such as a light-emitting diode, LED, and/or a laser diode. Further, the first light source may be a low pressure mercury plasma lamp or an excimer light source. The first light source may comprise a plurality of LEDs each of which emits the first light and the second light. By the term “plurality” is meant two or more.

The term “LED” as used in the context of the present invention implies any type of LED known in the art, such as inorganic LED(s), organic LED(s), polymer/polymeric LEDs, violet LEDs, blue LEDs, optically pumped phosphor coated LEDs, optically pumped nano-crystal LEDs. As used herein, the term “LED” can encompass a bare LED die arranged in a light mixing chamber, which may be referred to as a LED package. When UV-C light is used, the LED may be mounted in a cavity covered in a non-contact manner by an emission window made from quartz/fused silica.

The plurality of LEDs may comprise at least 10 LEDs, preferably at least 20 LEDs, more preferably at least 30 LEDs.

The disinfecting light module may comprise a plurality of first light sources. Each first light source of the plurality of first light sources may emit a first light in the UV wavelength range being same as or different from the UV wavelength range of the other first light sources. Further, each first light source of the plurality of first light sources may emit a second light in the visible wavelength range being same as or different from the visible wavelength range of the second light emitted by other first light sources. It is also conceivable that the second light emitted by each of the plurality of first light sources has a first color point x₁′, y₁′ being different from the first color point x₁, y₁ of the second light emitted by the other first light sources. Further, the disinfecting lighting device of the present invention may comprise a plurality of disinfecting light modules, each comprising one or several first light sources. The first light sources may be used together or may be operated individually depending on the type of microbiological species that needs to be deactivated.

The disinfecting lighting device according to the present invention further comprises at least one second light source configured to provide a third light in the visible wavelength range. Preferably, the at least one second light source does not emit UV light. The third light has a second color point x₂,y₂, being substantially equivalent to the first color point x₁,y₁.

The first color point x₁,y₁ and the second color point x₂,y₂ may fall within 7 SDCM. In other words, chromaticity coordinates of the second light and the third light both fall within 7 SDCM (or a “7-step MacAdam ellipse”). Preferably, the first color point x₁,y₁ and the second color point x₂,y₂ fall within 5 SDCM, more preferably within 4 SDCM, and most preferably within 3 SDCM.

The coordinates x₁,y₁, x₂,y₂ are defined in the CIE 1931 coordinate system. By the term “substantially equivalent” in the context of the present invention is meant that the following inequalities are fulfilled:

$$\vert {x}_{1} - {x}_{2} \vert < 0.05 \qquad ( 1 )$$

$$\vert {y}_{1} - {y}_{2} \vert < 0.05 \qquad ( 2 )$$

Preferably, the following inequalities are fulfilled:

$$\vert {x}_{1} - {x}_{2} \vert < 0.03 \qquad ( {1}^{′} )$$

$$\vert {y}_{1} - {y}_{2} \vert < 0.03 \qquad ( {2}^{′} )$$

More preferably, the following inequalities are fulfilled:

$$\vert {x}_{1} - {x}_{2} \vert < 0.02 \qquad ( {1}^{″} )$$

$$\vert {y}_{1} - {y}_{2} \vert < 0.02 \qquad ( {2}^{″} )$$

Most preferably, the following inequalities are fulfilled:

$$\vert {x}_{1} - {x}_{2} \vert < 0.01 \qquad ( {1}^{′′′} )$$

$$\vert {y}_{1} - {y}_{2} \vert < 0.01 \qquad ( {2}^{′′′} )$$

It is conceivable that the disinfecting lighting device comprises a plurality of second light sources, each providing a third light in the visible wavelength range being same as or different from the visible wavelength range of the third light emitted by the other second light sources. Further, it is also conceivable that the third light emitted by one of the plurality of second light sources has a second color point x₂′, y₂′ being different from the second color point x₂, y₂ of the third light emitted by the other second light sources. In the case when a plurality of first light sources is present, wherein the second light emitted by each first light source has a first color point x₁′, y₁′ being different from the first color point x₁, y₁ of the second light emitted by the other first light sources, each of the first light sources should have a corresponding second light source emitting a third light having a second color point x₂′, y₂′ being substantially equivalent to the first color point x₁′, y₁′.

The second light source adapted for, in operation, emitting visible light may for instance be provided as a solid state light source, e.g. white LEDs, as phosphor converted UV LEDs and/or blue LEDs, as RGB LEDs, or as a laser light source, or as a super luminescent diode.

The disinfecting lighting device further comprises an optical element arranged in a light-receiving relationship with the at least one disinfecting light module and the at least one second light source. The expression “in light-receiving relationship” in the context of the present invention means that the light emitted by the disinfecting light module and the second light source is transmitted through the optical element before it leaves the disinfecting lighting device.

According to the present invention, a portion of the optical element will thus receive light from the disinfecting light module, while the remaining portion of the optical element will receive light from the second light source. Since the second light emitted by the first light source and the third light emitted by the second light source have substantially equivalent color points, the entire optical element of the disinfecting lighting device will emit the light being perceived by the user as homogenous and pleasant. Therefore, the disinfecting lighting device of the present invention eliminates the disadvantage of bright and dark areas of the optical element.

The optical element of the present invention may be a light guide or a light mixing chamber. When the optical element is a light guide, it may have a light in-coupling portion, a light outcoupling portion and light outcoupling means. The third light may be coupled into the light guide at the light in-coupling portion. At least a portion of the third light may be light guided through a portion of the light guide based on total internal reflection. Subsequently, the third light may be coupled-out of the light guide at the light outcoupling portion by the light outcoupling means. The light guide may comprise quartz or polysiloxane.

The light guide may have a first major surface, a second major surface and at least one edge arranged between the first major surface and second major surface. The at least one edge of the light guide may constitute the light in-coupling portion. The first major surface may constitute the light outcoupling portion. The light outcoupling means may be a reflective dot pattern, which may be arranged on the second major surface.

In order to allow passage of the first light having disinfecting propertied through the optical element, the optical element should be light transmissive to the first light. The second light source may be arranged such that the optical element is side-lit by the third light. The light outcoupling pattern on the portion of optical element arranged in light-receiving relationship with the at least one disinfecting light module may be different from the light outcoupling pattern on the remaining portion of the optical element.

The wavelength of the second light may be within a first spectral range, and the wavelength of the third light may be within a second spectral range. Preferably, the first spectral range is different from the second spectral range.

Alternatively, the first spectral range of the second light may be the same as or partially overlapping with the second spectral range of the third light. In this case, the second light may have a first spectral distribution being different from a second spectral distribution of the third light. In other words, the second light and the third light may have different dominant peak wavelengths. The first and second spectral distribution may differ in the number of peaks and/or peak position, e.g. having a difference of at least 30 nm, preferably at least 50 nm in peak wavelength. The reason for such a difference may be that the first light source and the second light source are based on different technologies. Thus, the first light source may be based on a technology being different from solid state lighting, while the second light source may be based on solid state lighting, e.g. light emitting diodes (LED) or laser diodes. In particular, a phosphor converted LED may be used, such as a violet and/or blue LED comprising a phosphor layer. Alternatively, a combination of colored LEDs may be used such as violet and/or blue LEDs.

According to the present invention, the first light source during operation may be powered and depowered in a first intermittent manner. The term “powered” in the context of the present invention means switched on or dimmed up (increased intensity), while the term “depowered” means switched off or dimmed down (decreased intensity). Such intermittent mode of operation may be required by governmental regulations in order to minimize the dosage of the UV irradiation, while still achieving an adequate disinfection efficiency.

The first light source may thus be powered during a first time interval and depowered during a second time interval. The first time interval may be equivalent to the second time interval or may be different from the second time interval. Further, at least one of the first and the second time interval may vary during operation.

When the first light source is powered and depowered in a first intermittent manner, the user will be aware of such an operation, since when the first light source is depowered, no second light in the visible wavelength spectrum will be observed by the user. Such an intermittent manner of operation may thus be disturbing for the user, constantly observing a blinking light. Further, the user may have a concern whether the disinfecting lighting device provides sufficient disinfection efficiency.

In order to remedy these shortcomings, the second light source may during operation be powered and depowered in a second intermittent manner being inverse to the first intermittent manner. In other words, when the first light source is depowered, the second light source may be powered, emitting the third light having the same color point as the second light. Such an embodiment thus eliminates the disadvantage of bright and dark areas of the optical element as a function of time. At the moment the first light source is depowered, the second light source is powered, and a person present in the vicinity of the disinfecting lighting device has the impression that disinfection is continuously ongoing.

The disinfecting lighting device may further comprise a controller and a sensor, wherein the controller is arranged to power the second light source and to depower the first light source when presence of a subject is detected by the sensor. According to such an embodiment, a subject is prevented from exposure to the UV light, while getting an impression that the disinfection is ongoing when the subject is present in a room.

Alternatively, the second light source may operate in a continuous manner. Therefore, the user will not notice when the first light source is powered or depowered, since the disinfecting lighting device will constantly emit visible light being the second light, the third light or combinations thereof.

The first and the second intermittent manner may be a block wave or a sinusoidal wave.

The at least one disinfecting light module may comprise a first light mixing chamber comprising an inner cavity and a first light exit window. The at least one first light source may be arranged inside the inner cavity of the first light mixing chamber. The first light mixing chamber may thus be arranged to mix the first light and the second light. Further, the at least one second light source may be arranged inside the inner cavity of the first light mixing chamber. In such an embodiment, the first light mixing chamber may be arranged for mixing the first light, the second light and the third light. Such an embodiment offers the advantage of a compact lighting device providing good light mixing.

The first, second and third light may be emitted through the entire area of the first light exit window. The second light may have a first luminous flux LF₁, and the third light may have a second luminous flux LF₂. The first luminous flux may be substantially equivalent to the second luminous flux, i.e. 0.8<LF₁/LF₂<1.2.

The third light may be provided at a higher luminous flux LF₂ₐ When the first light source is powered, and at a lower luminous flux LF_{2b} when the first light source is depowered. When the second light and the third light are emitted through the entire area of the first light exit window, the following inequality may be valid:

$$0.8 < ( {LF}_{2 b} + {LF}_{1} ) / {LF}_{2 a} < 1.2$$

In such an embodiment, a constant light level is obtained as a function of time.

Alternatively, the first and second light may be emitted through a first portion of the first light exit window, while the third light may be emitted through a second portion of the first light exit window. Preferably, the first and the second portions constitute the entire area of the first light exit window. The second light has may have a first intensity I₁, and the third light may have a second intensity I₂. The first intensity may be substantially equivalent to the second intensity, i.e. 0.8<I₁/I₂<1.2. In such an embodiment, a homogenous lighting is obtained at any given time.

The reflectivity of the inner cavity of the first light mixing chamber may be at least 80%, more preferably at least 85%, most preferably at least 90%.

In the context of the invention, a light exit window is to be interpreted as any area, volume, or material which allow light to pass through it. The first light mixing chamber may be a hermetic light mixing chamber.

The first light mixing chamber may have any geometrical shape. The first light mixing chamber may be formed as a cuboid, where at least one face of the cuboid may act as a light exit window from where the first and the second light can be emitted. Preferably, the light mixing chamber comprises four light exit windows, such that the first and the second light can be emitted in four different directions.

The disinfecting lighting device may further comprise a second light mixing chamber comprising a second light exit window, wherein the at least one disinfecting light module, the optical element and the at least one second light source are arranged inside the second light mixing chamber. Further, when the first light mixing chamber is present, it may be arranged inside the second light mixing chamber. The disinfecting light module may thus be arranged inside the first light mixing chamber, which in turn is arranged inside the second light mixing chamber. The second light source may be arranged inside the first light mixing chamber, i.e. inside the disinfecting light module, or the second light mixing chamber, i.e. outside the disinfecting light module. The first and the second light exit windows may have different optical properties.

The disinfecting lighting device may comprise a reflector arranged between the optical element and the at least one disinfecting light module. In such an embodiment, the optical element may be arranged between the second light exit window and the reflector. The reflector may comprise at least one opening being aligned with the at least one disinfecting light module in order to allow passage of the first and the second light.

According to the present invention, the third light may have a purple color or a purplish white color. The third light may have wavelength within the purple wavelength range, i.e. the wavelength range from 400 nm to 430 nm. In such an embodiment, the second light source will provide a non-linear increase in the total intensity of the purple light when intensity of the first and the second lights is increased. Thus, the increase in disinfection intensity will be visually communicated to the user.

The disinfecting lighting device of the present invention may be a luminaire. The disinfecting lighting device may be configured to suspend from a ceiling of a room by a suspension arrangement or may be attached to the ceiling. Further, the disinfecting lighting device may be arranged at any other surface within the room, such as on a wall, on the floor or on a surface of a piece of furniture.

The disinfecting lighting device may further comprise a UV light sensor arranged for determining the intensity of output UV light and its distribution within the room.

Finally, a plurality of disinfecting lighting devices may be arranged at different locations within the same room and may further be in communication with each other.

Considering the above, the disinfecting lighting device of the present invention provides high safety level, allowing ongoing disinfection even when humans and/or animals are present in the area that is being disinfected. Moreover, the safety level of the disinfecting lighting device allows positioning the device anywhere in the room, such as a wall or a table, which in turn provides an efficient disinfection. Further, the disinfecting lighting device of the present invention has improved lighting perception.

### BRIEF DESCRIPTION OF THE DRAWINGS

Embodiments of the invention will now be described by way of example with reference to the accompanying drawings, of which:

FIGS. 1a-1b illustrate a disinfecting lighting device according to the present invention;

FIG. 2 shows another embodiment of the disinfecting lighting device, wherein the first light source is operated in a first intermittent manner:

FIG. 3 depicts an outcoupling pattern of the disinfecting lighting device:

FIG. 4 illustrates a cross-section view of the disinfecting lighting device comprising a reflector:

FIG. 5 illustrates non-linear increase in the purple light intensity:

FIGS. 6a and 6b show different embodiments of the first light mixing chamber:

FIGS. 7a-7c depict luminous flux or intensity plotted against time.

All the figures are schematic, not necessarily to scale, and generally only show parts which are necessary in order to elucidate embodiments of the present invention, wherein other parts may be omitted or merely suggested.

### DETAILED DESCRIPTION

The present invention will now be described hereinafter with reference to the accompanying drawings, in which exemplifying embodiments of the present invention are shown. The present invention may, however, be embodied in many different forms and should not be construed as limited to the embodiments of the present invention set forth herein; rather, these embodiments of the present invention are provided by way of example so that this disclosure will convey the scope of the invention to those skilled in the art. In the drawings, identical or similar reference numerals denote the same or similar components having a same or similar function, unless specifically stated otherwise.

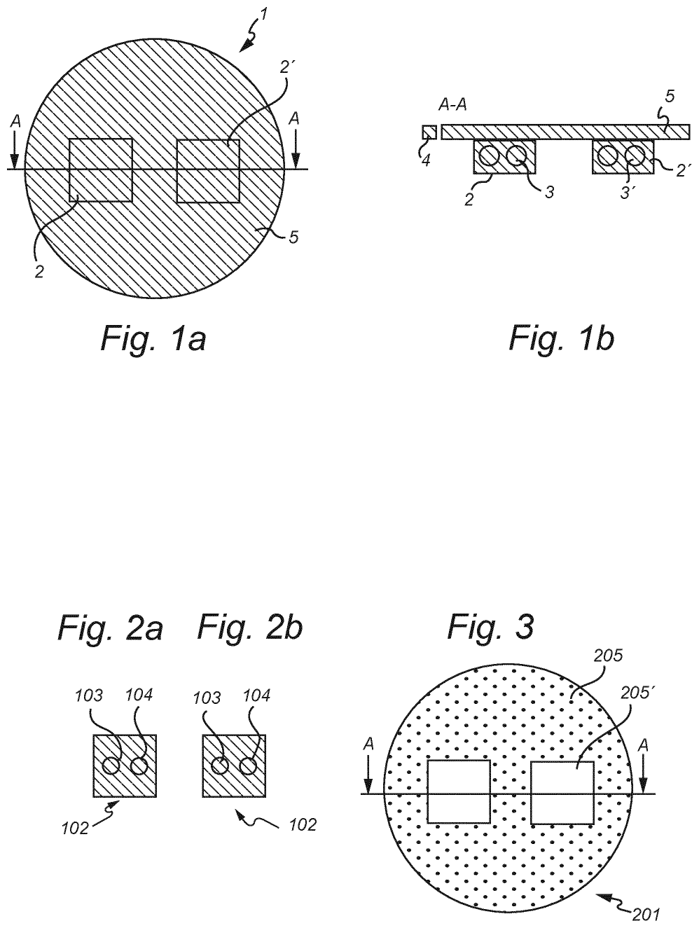

FIGS. 1a and 1b illustrate one embodiment of the disinfecting lighting device 1 according to the present invention. The disinfecting lighting device 1 comprises two disinfecting light modules 2, 2′ each comprising a first light source 3, 3′ providing a first light in the UV wavelength range and a second light in the visible wavelength range. The second light has a first color point x₁,y₁.

The disinfecting lighting device 1 further comprises one second light source 4 configured to provide a third light in the visible wavelength range. The third light has a second color point x₂,y₂, being substantially equivalent to the first color point x₁,y₁.

The disinfecting lighting device further comprises an optical element 5 arranged in a light-receiving relationship with the disinfecting light modules 2, 2′ and the second light source 4. It should be noted that in the embodiment depicted in FIGS. 1a and 1b the optical element 5 is side-lit by the third light emitted by the second light source.

According to the present invention, a portion of the optical element 5 will thus receive light from the disinfecting light modules 2, 2′, while the remaining portion of the optical element 5 will receive light from the second light source 4. Since the second light emitted by the first light sources 3, 3′ and the third light emitted by the second light source 4 have substantially equivalent color points, the entire optical element 5 of the disinfecting lighting device 1 will emit the light being perceived by the user as homogenous and pleasant. Therefore, the disinfecting lighting device of the present invention eliminates the disadvantage of bright and dark areas of the optical element 5.

FIGS. 2a and 2b illustrate a disinfecting light module 102 wherein the first light source 103 during operation is powered and depowered in a first intermittent manner. The second light source 104 is powered and depowered in a second intermittent manner being inverse to the first intermittent manner. In other words, when the first light source 103 is depowered, as shown in FIG. 2b, the second light source 104 may be powered, emitting the third light having the same color point as the second light. Therefore, the user will not notice when the first light source 103 is powered or depowered, since the disinfecting lighting device will constantly emit visible light being the second light, the third light or combinations thereof.

As may be seen in FIG. 3, the light outcoupling pattern on the portion of optical element 205′ arranged in light-receiving relationship with the at least one disinfecting light module is different from the light outcoupling pattern on the remaining portion of the optical element 205.

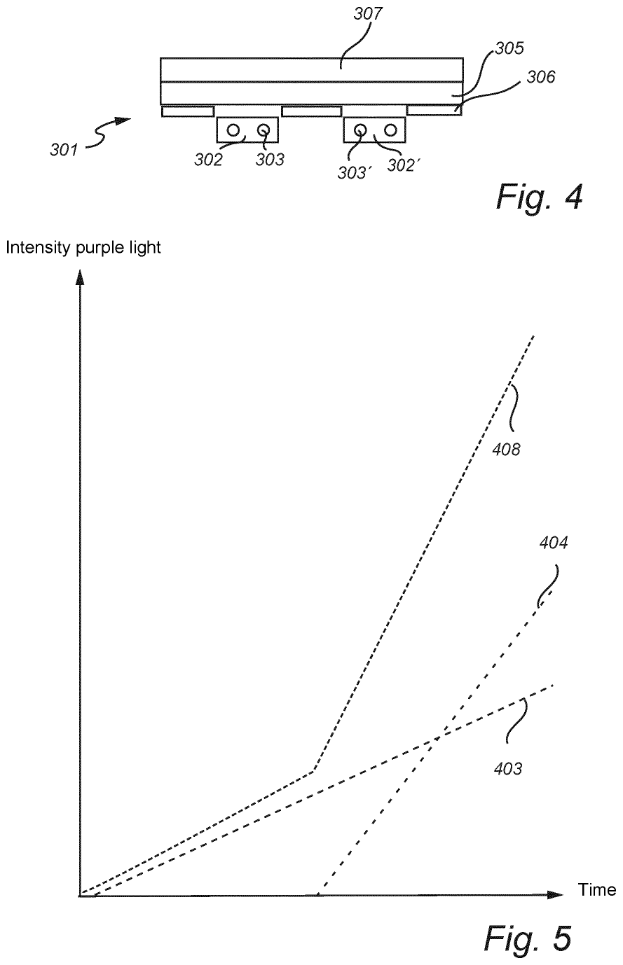

FIG. 4 depicts a disinfecting lighting device 301 comprising a reflector 306 arranged between the optical element 305 and the disinfecting light modules 302, 302′. The optical element 305 is arranged between the second light exit window 307 and the reflector 306. The reflector 306 comprises two openings being aligned with the two disinfecting light modules 302, 302′.

FIG. 5 depicts intensity of purple light plotted against time. The second light source 404 will provide a non-linear increase in the total intensity of the purple light 408 when intensity of the first and the second lights emitted by the first light source 403 is increased. Thus, the increase in disinfection intensity will be visually communicated to the user.

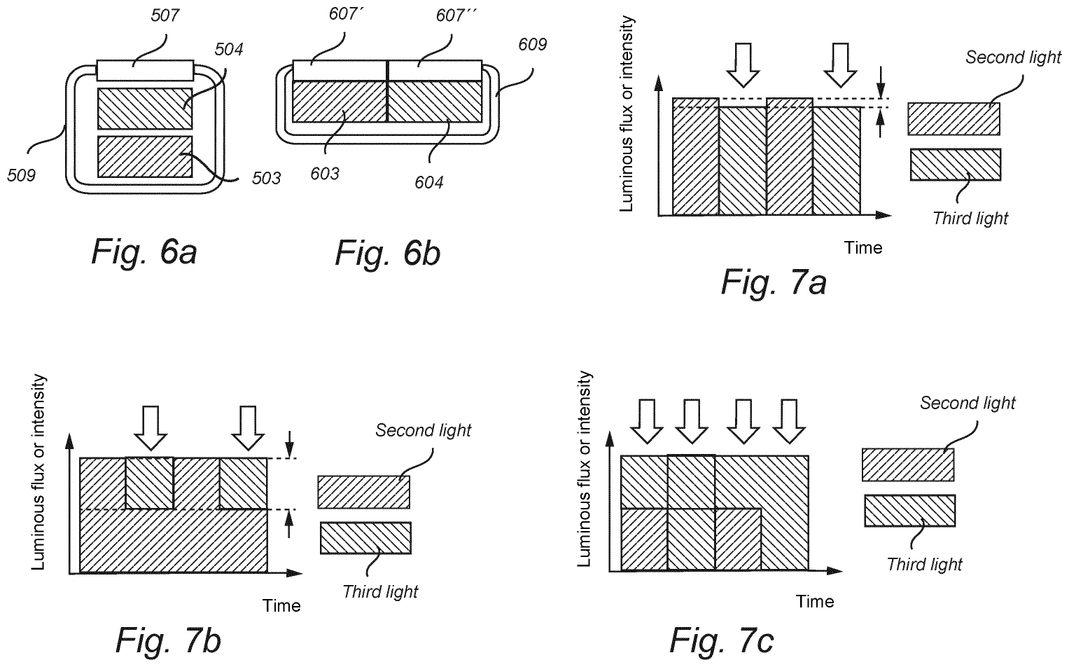

FIG. 6a depicts a first light mixing chamber 509 comprising a first light source 503 and a second light source 504. As may be seen in FIG. 6a, the first, second and third light will be emitted through the entire area of the first light exit window 507. In this case, the first luminous flux LF₁ of the second light and the second luminous flux LF₂ of the third light should satisfy the inequality 0.8<LF₁/LF₂<1.2. i.e. these values should be substantially equivalent, as shown in FIGS. 7a-7c.

FIG. 6b illustrates a first light mixing chamber 609 comprising a first light source 603 and a second light source 604. As may be seen in FIG. 6b, the first and second light will be emitted through a first portion 607′ of the first light exit window, while the third light will be emitted through a second portion 607″ of the first light exit window. Accordingly, the second light has may have a first intensity I₁, and the third light may have a second intensity I₂. The first intensity may be substantially equivalent to the second intensity, i.e. 0.8<I₁/I₂<1.2, as illustrated in FIGS. 7a-7c.

Although the present invention has been described with reference to various embodiments, those skilled in the art will recognize that changes may be made without departing from the scope of the invention. It is intended that the detailed description be regarded as illustrative and that the appended claims including all the equivalents are intended to define the scope of the invention. While the present invention has been illustrated in the appended drawings and the foregoing description, such illustration is to be considered illustrative or exemplifying and not restrictive: the present invention is not limited to the disclosed embodiments. Other variations to the disclosed embodiments can be understood and effected by those skilled in the art in practicing the claimed invention, from a study of the drawings, the disclosure, and the appended claims. In the appended claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A disinfecting lighting device arranged to emit device light, said disinfecting lighting device comprising:
at least one disinfecting light module comprising at least one first light source providing a first light in the UV wavelength range and a second light in the visible wavelength range, said second light having a first color point x₁,y₁;
at least one second light source configured to provide a third light in the visible wavelength range, said third light having a second color point x₂,y₂;
an optical element arranged in a light-receiving relationship with said at least one disinfecting light module and said second light source;
wherein said first color point x₁,y₁ and said second color point x₂,y₂ are substantially equivalent.

2. The disinfecting lighting device according to claim 1, wherein the following equations are fulfilled:
$$\vert {x}_{1} - {x}_{2} \vert < 0.05 \qquad ( 1 )$$
$$\vert {y}_{1} - {y}_{2} \vert < 0.05 \qquad ( 2 )$$

3. The disinfecting lighting device according to claim 1, wherein said second light has a first spectral distribution and said third light has a second spectral distribution, and wherein said first spectral distribution is different from said second spectral distribution.

4. The disinfecting lighting device according to claim 1, wherein said optical element is a light guide, said light guide having a light in-coupling portion, a light outcoupling portion and light outcoupling means, wherein at least a first portion of said third light is coupled into the light guide at said light in-coupling portion, at least a second portion of the third light is light guided through a portion of the light guide based on total internal reflection, said at least second portion of said third light is coupled-out of said light guide at said light outcoupling portion by said light outcoupling means.

5. The disinfecting lighting device according to claim 1, wherein said optical element is light transmissive to said first light.

6. The disinfecting lighting device according to claim 1, wherein said at least one disinfecting light module comprises a first light mixing chamber comprising an inner cavity and a first light exit window, and wherein said at least one first light source is arranged inside said inner cavity of said first light mixing chamber.

7. The disinfecting lighting device according to claim 6, wherein said at least one second light source is arranged inside said inner cavity of said first light mixing chamber.

8. The disinfecting lighting device according to claim 1, wherein said first light source during operation is powered and depowered in a first intermittent manner.

9. The disinfecting lighting device according to claim 8, wherein said second light source during operation is powered and depowered in a second intermittent manner being inverse to said first intermittent manner.

10. The disinfecting lighting device according to claim 9, wherein said second light has a first luminous flux LF₁, said third light has a second luminous flux LF₂, wherein 0.8<LF₁/LF₂<1.2.

11. The disinfecting lighting device according to claim 9, wherein said third light is provided at a higher luminous flux LF₂ₐ when said first light source is powered, and at a lower luminous flux LF_{2b} when said first light source is depowered, wherein 0.8<(LF_{2b}+LF₁)/LF₂ₐ<1.2.

12. The disinfecting lighting device according to claim 1, wherein said disinfecting lighting device further comprises a second light mixing chamber comprising a second light exit window, and wherein said at least one disinfecting light module, said optical element and said at least one second light source are arranged inside said second light mixing chamber.

13. The disinfecting lighting device according to claim 10, wherein said disinfecting lighting device comprises a reflector arranged between said optical element and said at least one disinfecting light module, wherein said optical element is arranged between said second light exit window and said reflector, and wherein said reflector comprises at least one opening being aligned with said at least one disinfecting light module.

14. The disinfecting lighting device according to claim 1, wherein said disinfecting lighting device further comprises a controller and a sensor, wherein said controller is arranged to power said second light source and to depower said first light source when presence of a subject is detected by said sensor.

15. The disinfecting lighting device according to claim 1, wherein said second light and said third light has a purple color or a purplish white color.
